(19) United States
(12) Patent Application Publication
(10) Pub. No.: US 2012/0035405 A1
(43) Pub. Date: Feb. 9, 2012

(54) METHOD FOR ENHANCED GAS HYDRATE PERMEABILITY

(75) Inventors: Keith C. Hester, Tulsa, OK (US); James J. Howard, Tulsa, OK (US)

(73) Assignee: CONOCOPHILLIPS COMPANY, Houston, TX (US)

(21) Appl. No.: 13/189,229

(22) Filed: Jul. 22, 2011

Related U.S. Application Data
(60) Provisional application No. 61/371,824, filed on Aug. 9, 2010.

(51) Int. Cl. C07C 7/00; E21B 43/22; E21B 43/16

(52) U.S. Cl. 585/833; 166/305.1; 585/841; 585/860; 585/868

(57) ABSTRACT

The present invention relates to an improved method for recovering hydrocarbons trapped in hydrate formations.

### CROSS-REFERENCE TO RELATED APPLICATIONS

This application claims priority benefit under 35 U.S.C. Section 119(e) to U.S. Provisional Patent Ser. No. 61/371,824 filed on Aug. 9, 2010, the entire disclosure of which is incorporated herein by reference.

### FIELD OF THE INVENTION

The present invention relates to an improved method for recovering hydrocarbons trapped in hydrate formations.

### BACKGROUND OF THE INVENTION

A number of hydrocarbons, especially lower boiling-point light hydrocarbons, in formation fluids or natural gas, are known to form hydrates in conjunction with the water present under a variety of conditions—particularly at a combination of lower temperatures and higher pressures. The hydrates are solid crystailline compounds which co-exist with the surrounding formation or natural gas fluids. Any solids in a formation or natural gas fluid are at least a nuisance for production, handling, and transport of these fluids. It is not uncommon for solid hydrates to cause plugging and/or blockage of pipelines or transfer lines or other conduits, valves and/or safety devices and/or other equipment, resulting in shutdown, loss of production, and risk of explosion or unintended release of hydrocarbons into the environment either on-land or off-shore. Accordingly, hydrocarbon hydrates have been of substantial interest as well as concern to many industries.

Gas hydrates are in a class of compounds know as clathrates, and are also referred to as inclusion compounds. Clathrates consist of cage structures formed between a host molecule and a guest molecule. Gas hydrates are generally composed of crystals formed by water host molecules surrounding the hydrocarbon guest molecules. The smaller or lower-boiling hydrocarbon molecules, particularly C₁ (methane) to C₄ hydrocarbons and their mixtures, form in hydrate or clathrate crystals under a wide range of production conditions. Even certain non-hydrocarbons such as carbon dioxide and hydrogen sulfide are known to form hydrates under the proper conditions.

Conventional methods of natural gas extraction from gas hydrates involve heating and/or depressurizing the gas hydrates in order to release the natural gas. However, there are two concerns with these conventional methods. First, these methods may require a large amount of energy to be added to the system, especially in heating methods, resulting in a high cost of extraction. Second, these methods destabilize hydrate formations causing the hydrate to dissociate, which can lead to the destabilization and/or collapse of sediments containing hydrates and other nearby subterranean reservoirs. Because gas hydrates are often located near oil and natural gas deposits, such instability during extraction can result in difficulties with the extraction of oil and natural gas.

Alternatively, releasing agent exchange methods have been shown possible in the laboratory for the extraction of natural gas hydrates. The releasing agent employed in the exchange process may be a hydrate-forming agent, which is thermodynamically more stable under system conditions than the natural gas hydrate. Injection of a suitable releasing agent has been shown to release the natural gas in the hydrate while simultaneously sequestering the releasing agent in the hydrate cages. This process only requires the injection of a releasing agent and is therefore less energy intensive then conventional methods. In addition, because the releasing agent enters the hydrate and exchanges with natural gas, bulk hydrate dissociation is not thought to occur, mitigating the destabilization risk posed by hydrate dissociation during production.

However, if free water is available in the hydrate formation, injection of the releasing agent can cause secondary hydrate formation, i.e., pure hydrate composed of the releasing agent. This secondary hydrate formation can lead to a reduction in permeability, limiting the applicability of the releasing agent exchange method. Therefore, a need exists for a technique which enhances permeability and inhibits additional hydrate formation in a controlled manner during extraction of natural gas from gas hydrates, especially with a method similar to releasing agent exchange.

### SUMMARY OF THE INVENTION

In an embodiment, a method for producing hydrocarbons from a hydrate-bearing subterranean reservoir includes: (a) introducing a releasing agent into the subterranean reservoir, wherein the releasing agent liquid carbon dioxide; (b) injecting a reagent into the subterranean reservoir, wherein the reagent is intermittently injected into the subterranean channel, wherein the reagent is nitrogen; and (c) recovering the released hydrocarbons from the subterranean reservoir.

In another embodiment, a method for producing hydrocarbons from a subterranean reservoir includes: (a) introducing a releasing agent into the subterranean reservoir; (b) injecting a reagent into the subterranean reservoir; and (c) recovering the released hydrocarbons from the subterranean reservoir.

In further embodiment, a method for releasing hydrocarbons from a gas hydrate, said gas hydrate comprising a hydrocarbon bound with solid-state water, the method includes: (a) substituting a releasing agent for the hydrocarbon to thereby release the hydrocarbon from the solid-state water without melting the gas hydrate with the assistance of a reagent to capture any free water present, thereby providing a substituted hydrate comprising the releasing agent bound with the solid-state water.

### BRIEF DESCRIPTION OF THE DRAWINGS

The invention, together with further advantages thereof, may best be understood by reference to the following description taken in conjunction with the accompanying drawings in which:

FIG. 1 is a schematic diagram showing an embodiment.

### DETAILED DESCRIPTION OF THE INVENTION

Reference will now be made in detail to embodiments of the present invention, one or more examples of which are illustrated in the accompanying drawing. Each example is provided by way of explanation of the invention, not as a limitation of the invention. It will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations that come within the scope of the appended claims and their equivalents.

Methods and compositions have been discovered for inhibiting, retarding, mitigating, reducing, controlling and/or delaying formation of hydrocarbon hydrates or agglomerates of hydrates in hydrocarbon recovery operations. These methods may be applied to prevent or reduce or mitigate plugging of annular spaces, pipes, transfer lines, valves, and other places or equipment downhole where hydrocarbon hydrate solids may form under conditions conducive to their formation or agglomeration.

To improve permeability and inhibit additional hydrate formation, a releasing agent and a reagent are employed to prevent natural gas hydrate buildup within a subterranean reservoir. As used herein, a natural gas hydrate is a crystalline clathrate hydrate containing hydrocarbons such as a major component of methane, and trace amounts of ethane, propane and butane. As used herein, the subterranean reservoir may include porous rock or sediments that are associated with the proper pressure and temperature conditions necessary to form natural gas hydrates.

A releasing agent is contacted with the natural gas hydrate, resulting in the releasing agent spontaneously (i.e., without the need for added energy) replacing the gas within the hydrate without requiring a significant change in the temperature, pressure, or volume of the hydrate. As used herein, the releasing agent is a compound that forms a more thermodynamically stable hydrate structure than the gas originally contained within the hydrate structure. The releasing agent is selected from a group consisting of stable hydrate-forming molecules which could include but not limited to carbon dioxide, xenon, hydrogen sulfide, and mixtures thereof. In an embodiment, the releasing agent is carbon dioxide. In another embodiment, the releasing agent is liquid. In yet another embodiment, the releasing agent is liquid carbon dioxide.

However, if any free water is present in the hydrate deposit, the injection of the releasing agent could potentially lead to the formation of additional hydrates. Introducing a reagent, a non-hydrate forming gas under reservoir conditions, can assist in altering the stability conditions of the hydrate and subsequently promote dissociation and enhance permeability without requiring a significant change in temperature, pressure and composition of the hydrate. The reagent utilized is a non-hydrate forming gas under reservoir conditions. Examples of non-hydrate forming gases under reservoir conditions include, but are not limited to: nitrogen, helium, hydrogen, argon, krypton, neon and mixtures thereof. Furthermore, reagent mixtures can include air or flue gas. Injection of the reagent induces hydrate dissociation until the mixture composition of reagent and released gas is thermodynamically stable. The volumes and rates of reagent injection are determined by comparing the amount/composition of the hydrate required for dissociation to achieve desired reservoir permeability with the amount of reagent needed to achieve that level of dissociation before the system returns to thermodynamic stability

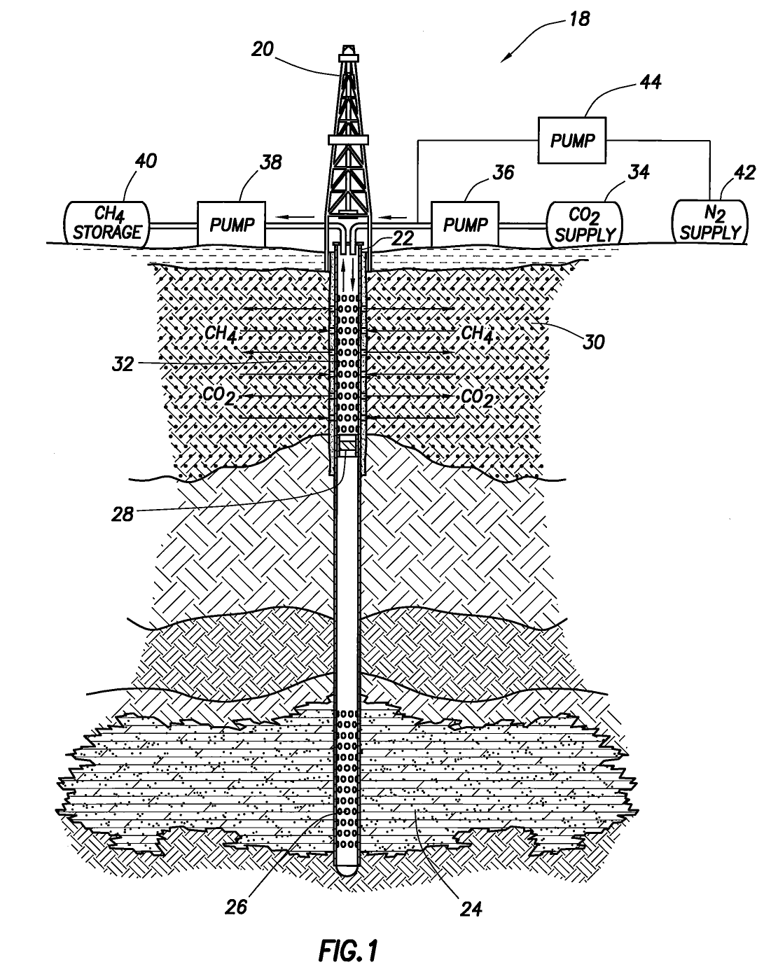

FIG. 1 illustrates an oil or natural gas well 18 for facilitating improved permeability and inhibiting additional hydrate formation. Well 18 generally includes a superstructure 20 and a casing 22. Well 18 was previously used to produce oil and/or gas from a subterranean reservoir 24 via lower perforations 26 in casing 22. In an embodiment, the well id drilled for hydrate production. After production of the oil and/or gas from subterranean reservoir 24 has been completed, a plug 28 is inserted into casing 22 above perforations 26 and immediately below a gas hydrate formation 30. The casing 22 and the perforations 26 create a subterranean channel, which provides access to the hydrates in question. In an embodiment, the gas hydrate is a methane hydrate. Upper perforations 32 are created in casing 22 above plug 28 and proximate to hydrate formation 30. Once plug 28 and perforations 32 are in place, liquid carbon dioxide, i.e., the releasing agent, from carbon dioxide supply 34 can be introduced into casing 22 via a carbon dioxide pump 36. The nitrogen gas, i.e., the reagent, can then be introduced from a nitrogen gas supply 42 into casing 22 via nitrogen gas pump 44. In an embodiment, the reagent is introduced into a hydrate-bearing formation within a subterranean reservoir prior to the releasing agent. In another embodiment, the reagent is introduced into the hydrate-bearing formation within a subterranean reservoir during the injection of the releasing agent. In a further embodiment, the reagent is interlaced with the releasing agent. The injection of the nitrogen gas is controlled in an effort to open up the hydrate-filled reservoir for more efficient liquid carbon dioxide injection. The nitrogen gas and liquid carbon dioxide introduced into casing 22 are then discharged from casing 22, either independently or collectively, and into hydrate-bearing formation 30 via upper perforations 32. As described in detail above, when the carbon dioxide contacts the hydrates of hydrate-bearing formation 30, the carbon dioxide molecules are spontaneously substituted for the methane molecules in the hydrate structure, thereby releasing free methane gas without melting hydrate-bearing formation 30.

In order to successfully introduce the reagent into the reservoir, the injection pressure must be greater than pore pressure. Furthermore, the reagent injection pressure could be less than or greater than the formation lithostatic pressure. Injection pressure should be less than lithostatic pressure (or a value close to this pressure) in order to avoid fracturing of the reservoir. If the reagent injection pressure is greater than the lithostatic pressure, then the injected reagent could fracture the hydrate-bearing formation layer and stimulate the permeability of the well, if desired. The volumes and rates of reagent injection are determined by comparing the amount/composition of the hydrate needed to be dissociated to achieve desired reservoir permeability and the amount of reagent need to achieve that level of dissociation before the system returns to thermodynamic stability.

The released free methane gas flows back towards casing 22 and enters casing 22 via perforations 32. The released methane gas can then be evacuated from casing 22 using a methane pump 38. The recovered methane gas can be stored on site in methane storage 40 or can be immediately transported off site for further processing. The carbon dioxide used to replace/release the methane is permanently sequestered in the subterranean reservoir.

In an alternate embodiment, injection of the releasing agent and reagent can occur during the releasing agent exchange process. One example would be to monitor well pressure during reagent injection until permeability is reduced to a pre-determined value. Injection of the reagent would follow to increase permeability and releasing agent injection could then resume. This process could occur indefinitely over the production cycle of the subterranean reservoir.

The preferred embodiment of the present invention has been disclosed and illustrated. However, the invention is intended to be as broad as defined in the claims below. Those skilled in the art may be able to study the preferred embodiments and identify other ways to practice the invention that are not exactly as described in the present invention. It is the intent of the inventors that variations and equivalents of the invention are within the scope of the claims below and the description, abstract and drawings not to be used to limit the scope of the invention.

1. A method for producing hydrocarbons from a hydrate-bearing subterranean reservoir comprising:
a. introducing a releasing agent into the subterranean reservoir, wherein the releasing agent liquid carbon dioxide;
b. injecting a reagent into the subterranean reservoir, wherein the reagent is intermittently injected into the subterranean channel, wherein the reagent is nitrogen; and
c. recovering the released hydrocarbons from the subterranean reservoir.

2. The method according to claim 1, wherein the reagent is injected simultaneously with the releasing agent.

3. The method according to claim 2, wherein the reagent is interlaced with the releasing agent.

4. A method for producing hydrocarbons from a subterranean reservoir comprising:
a. introducing a releasing agent into the subterranean reservoir;
b. injecting a reagent into the subterranean reservoir; and
c. recovering the released hydrocarbons from the subterranean reservoir.

5. The method according to claim 4, wherein the reagent is intermittently injected into the subterranean channel.

6. The method according to claim 4, wherein the reagent is injected simultaneously with the releasing agent.

7. The method according to claim 6, wherein the reagent is interlaced with the releasing agent.

8. The method according to claim 4, wherein the reagent is a non-hydrate forming gas under subterranean reservoir conditions.

9. The method according to claim 8, wherein the reagent is selected from a group consisting of nitrogen, air, helium, oxygen, chlorine, hydrogen, argon, krypton, neon and mixtures thereof.

10. The methane according to claim 9, wherein the reagent is nitrogen.

11. The method according to claim 4, wherein the releasing agent is selected from a group consisting of carbon dioxide, nitrous oxide, and mixtures thereof.

12. The method according to claim 11, wherein the releasing agent is carbon dioxide.

13. The method according to claim 11, wherein the releasing agent is liquid.

14. The method according to claim 13, wherein the releasing agent is liquid carbon dioxide.

15. The method according to claim 4, wherein the releasing agent is a liquid.

16. A method for releasing hydrocarbons from a gas hydrate, said gas hydrate comprising a hydrocarbon bound with solid-state water, said method comprising:
a. substituting a releasing agent for the hydrocarbon to thereby release the hydrocarbon from the solid-state water without melting the gas hydrate with the assistance of a reagent to capture any free water present, thereby providing a substituted hydrate comprising the releasing agent bound with the solid-state water.

17. The method according to claim 16, wherein the substituted hydrate is more stable than the gas hydrate.

18. The method according to claim 16, wherein the gas hydrate is a methane hydrate.

19. The method according to claim 16, wherein the hydrocarbon is methane.

20. The method according to claim 16, wherein the reagent is a non-hydrate forming gas.

21. The method according to claim 20, wherein the reagent is selected from a group consisting of nitrogen, air, helium, oxygen, chlorine, hydrogen, argon, krypton, neon and mixtures thereof.

22. The method according to claim 21, wherein the hydrate is nitrogen gas.

23. The method according to claim 16, wherein the releasing agent is selected from a group consisting of carbon dioxide, nitrous oxide, and mixtures thereof.

24. The method according to claim 23, wherein the releasing agent comprises carbon dioxide.

25. The method according to claim 23, wherein the releasing agent is liquid.

26. The method according to claim 25, wherein the releasing agent is liquid carbon dioxide.

27. The method according to claim 16, wherein the releasing agent is in liquid phase when contacted with the gas hydrate.
